(19) United States
(12) Patent Application Publication
(10) Pub. No.: US 2025/0312091 A1
(43) Pub. Date: Oct. 9, 2025

(54) ABLATION CATHETERS WITH ELECTROPLATED HEATING ELEMENT TO TREAT VARICOSE VEINS

(71) Applicant: Boston Scientific Medical Device Limited, Galway (IE)

(72) Inventors: Yinghua Wang, Shanghai (CN); Mengxiang Luo, Shanghai (CN); Mingfeng Frank Xie, Shanghai (CN); Cheng Zhang, Shanghai (CN); Longsheng Cai, Shanghai (CN); Linshanzi Pan, Shanghai (CN)

(21) Appl. No.: 18/881,626

(22) PCT Filed: Jul. 13, 2023
(86) PCT No.: PCT/EP2023/069528

§ 371 Date: Jan. 6, 2025

(30) Foreign Application Priority Data
Jul. 14, 2022 (CN) 202210834216.2

(51) Int. Cl. A61B 18/14; A61B 18/00

(52) U.S. Cl. CPC A61B 18/1492; A61B 2018/00077; A61B 2018/0016; A61B 2018/00404; A61B 2018/00577; A61B 2018/00642; A61B 2018/00773

(57) ABSTRACT

At least some embodiments of the present disclosure are directed to a catheter for use in varicose vein treatment including a handle, an elongated shaft connected to the handle, and a heating element disposed near the distal end of the shaft. In some embodiments, the heating element includes an electrically conductive trace defining a plurality of parallel segments arranged along a length of the tubular polymeric member, and an arcuate segment disposed between and connecting the parallel segments.

### TECHNICAL FIELD

The present disclosure pertains to medical devices, systems, and methods for providing a therapeutic heat treatment. More particularly, the present disclosure pertains to medical devices, systems and methods for providing therapeutic heat treatments to venous diseases.

### BACKGROUND

Therapeutic heat treatment can be used to treat a wide variety of medical conditions such as tumors, fungal growth, etc. Heat treatments can be used for treating medical conditions alongside other therapeutic approaches or as a standalone therapy. Heat treatment provides localized heating and thus does not cause any cumulative toxicity in contrast to other treatment methods such as drug-based therapy, for example.

One exemplary clinical application of therapeutic heat treatment is in the treatment of chronic venous diseases such as varicose veins, which may become enlarged and/or tortuous due to one or more pathological conditions. Application of sufficient thermal energy via an intravascular device can treat varicose veins by constricting or occluding the target veins.

There is a continuing need for improved devices and methods to provide focused, controlled thermal energy for thermally treating chronic venous conditions such as varicose veins while minimizing or eliminating effects on surrounding healthy tissue.

### SUMMARY

In Example 1, a device for treating varicose veins includes a catheter including an elongated shaft having a proximal end and a distal end, the shaft being sized and configured such that the distal end can be inserted into a target blood vessel; and an ablation element disposed near the distal end of the elongated shaft. In some embodiments, the ablation element includes a tubular polymeric member connected to the elongated shaft; and a heating element disposed on the tubular polymeric member, the heating element comprising an electrically conductive trace defining a plurality of parallel segments arranged along a length of the tubular polymeric member and one or more arcuate segments, at least one of the one or more arcuate segments disposed between and connecting two adjacent parallel segments of the plurality of parallel segments.

In Example 2, the device of Example 1, wherein the electrically conductive trace has a serpentine shape defined by the plurality of parallel segments and the one or more arcuate segments, wherein the parallel segments are longitudinally or circumferentially spaced from one another on the tubular polymeric member.

In Example 3, the device of Example 2, wherein the plurality of parallel segments extend longitudinally along the tubular polymeric member and are circumferentially spaced from one another.

In Example 4, the device of Example 2, wherein the plurality of parallel segments extend circumferentially about the tubular polymeric member and are longitudinally spaced from one another.

In Example 5, the device of Example 1, wherein the plurality of parallel segments are helically wound about the tubular polymeric member.

In Example 6, the device of any of Examples 1-5, wherein the heating element is disposed on an outer surface of the tubular polymeric member.

In Example 7, the device of any of Examples 1-5, wherein at least a part of the heating element is disposed on an inner surface of the tubular polymeric member.

In Example 8, the device of any of Examples 1-7, wherein the heating element comprises a Ni—Cr alloy.

In Example 9, the device of any of Examples 1-7, wherein the heating element comprises a carbon film.

In Example 10, the device of any of Examples 1-9, herein the plurality of parallel segments are equally spaced apart.

In Example 11, the device of Example 8, wherein the Ni—Cr alloy is electroplated or spayed onto the tubular polymeric member.

In Example 12, the device of Example 9, wherein the carbon film is electroplated or sprayed onto the tubular polymeric member.

In Example 13, a system for treating varicose veins includes the device of any of Examples 1-12; an energy generator connected to the elongated catheter and configured to generate an electric signal; and a controller operatively connected to the energy generator to control the generation of the electric signal.

In Example 14, the system of Example 13, wherein the heating element is electrically coupled to the energy generator.

In Example 15, the system of Example 13, wherein the device includes one or more pressure sensors configured to generate an output signal indicative of a pressure applied thereto, and wherein the controller is configured to adjust the electric signal to be delivered to the heating element based upon the output signal.

While multiple embodiments are disclosed, still other embodiments of the present invention will become apparent to those skilled in the art from the following detailed description, which shows and describes illustrative embodiments of the invention. Accordingly, the drawings and detailed description are to be regarded as illustrative in nature and not restrictive.

### BRIEF DESCRIPTION OF THE DRAWINGS

FIG. 1 is a schematic illustration of an exemplary ablation device for treating chronic venous diseases, e.g., varicose veins, according to an embodiment of the present disclosure.

FIG. 2A is a schematic illustration of an exemplary ablation catheter including a connector for treating chronic venous diseases, e.g., varicose veins, according to an embodiment of the present disclosure.

FIG. 2B is a schematic cross-sectional view of a connector of the exemplary ablation catheter of FIG. 2A, according to embodiments of the present disclosure.

FIGS. 3A and 3B are schematic elevation and partial blown-up views, respectively, of the distal end portion of an ablation catheter, according to embodiments of the present disclosure.

FIGS. 4A-4C are phantom schematic elevation, partial blown-up, and cutaway views, respectively, of the distal end portion of an ablation catheter, according to embodiments of the present disclosure.

FIG. 5 is a schematic illustration of the distal end portion of an ablation catheter, according to embodiments of the present disclosure.

FIGS. 6A-6B are schematic illustrations of a portion of an ablation catheter for use in a target blood vessel in a patient for treatment of varicose veins, according to embodiments of the present disclosure.

While the invention is amenable to various modifications and alternative forms, specific embodiments have been shown by way of example in the drawings and are described in detail below. The intention, however, is not to limit the invention to the particular embodiments described. On the contrary, the invention is intended to cover all modifications, equivalents, and alternatives falling within the scope of the invention as defined by the appended claims.

### DETAILED DESCRIPTION

The following detailed description is exemplary in nature and is not intended to limit the scope, applicability, or configuration of the invention in any way. Rather, the following description provides some practical illustrations for implementing exemplary embodiments of the present invention. Examples of constructions, materials, and/or dimensions are provided for selected elements. Those skilled in the art will recognize that many of the noted examples have a variety of suitable alternatives.

Therapeutic heat treatment can be used to treat a wide variety of medical conditions including chronic venous diseases such as varicose veins, which may become enlarged and/or tortuous due to one or more pathological conditions. Application of sufficient thermal energy via an intravascular device can treat varicose veins by constricting or occluding the target veins.

An exemplary catheter for use in varicose vein treatment may include a handle, an elongated shaft connected to the handle, and a heating element disposed near the distal end of the shaft. In some embodiments, the heating element may receive currents (e.g., alternating currents, direct currents) delivered by an energy generator to generate and deliver thermal ablative energy. In certain embodiments, the heating element may receive electrical signals (e.g., radiofrequency alternating currents) generated by an energy generator to generate and deliver radiofrequency ablative energy.

As mentioned above, there is a continuing need for improved devices and methods to provide focused, controlled thermal energy for thermally treating chronic venous conditions such as varicose veins while minimizing or eliminating effects on surrounding healthy tissue. For example, a degree of flexibility is desired on the catheter used to treat target blood vessel to minimize potential undesirable harm to vessel walls during treatment. Certain embodiments of the present disclosure can improve heating therapy efficiency while maintaining the degree of flexibility of the catheter. Alternative ways of providing thermal energy for the treatment is also desired for improved and diversified treatment methods.

Some embodiments of the present disclosure describe a catheter with an elongated shaft and an ablation element disposed near the distal end of the elongated shaft, where the ablation element includes a tubular polymeric member connected to the elongated shaft and a heating element disposed on the tubular polymeric member and operatively connected to the energy generator. In some embodiments, the heating element includes an electrically conductive trace defining a plurality of parallel segments arranged along a length of the tubular polymeric member, and one or more arcuate segment disposed between and connecting the parallel segments. The tubular polymeric member may be made of flexible materials, and after electroplating or spraying, the tubular polymeric member may maintain its flexibility to deliver effective treatment to the target treatment site while minimizing potential undesirable harm to vessel walls.

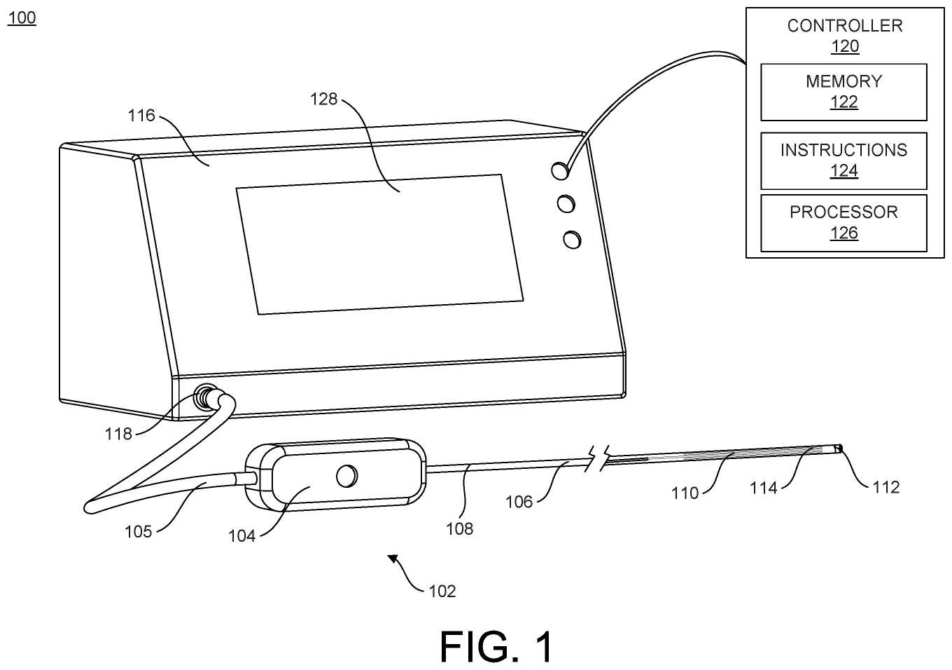

FIG. 1 is a schematic illustration of an exemplary ablation device 100 for treating chronic venous diseases, e.g., varicose veins, according to an embodiment of the present disclosure. The ablation device 100 includes an ablation catheter 102 including a handle 104, an elongated shaft 106 having a proximal end 108 and a distal end portion 110 terminating at a distal end 112, and an ablation element 114 disposed near the distal end 112 of the elongated shaft 106. The shaft 106 is sized and configured such that the distal end 112 may be inserted into a target blood vessel. The ablation element 114 is configured to deliver ablative energy (e.g., radiofrequency energy, thermal energy) to walls of a target blood vessel.

The ablation device 100 may include an energy generator 116 electrically coupled to the handle 104 via a connector 118 and configured to generate energy by delivering an electric signal (e.g., currents, radiofrequency alternating currents). A controller 120 is operatively connected to the energy generator 116 to control the generation of the electric signal. The controller 120 can be implemented using firmware, integrated circuits, and/or software modules that interact with each other or are combined together. For example, the controller 120 may include memory 122 storing computer-readable instructions/code 124 for execution by a processor 126 (e.g., microprocessor) to perform aspects of embodiments of methods discussed herein.

According to certain embodiments, the ablation element 114 employs structural features and/or components to improve the clinical performance as well as enhance the manufacturability of the ablation catheter 102. In some embodiments, the ablation element 114 may include a tubular polymeric member connected to the elongated shaft 106 and a heating element disposed on the tubular polymeric member and operatively connected to the energy generator 116. In some embodiments, as will be discussed in more details herein, the heating element includes an electrically conductive trace defining a plurality of parallel segments arranged along a length of the tubular polymeric member, and one or more arcuate segment disposed between and connecting the parallel segments.

In some embodiments, the controller 120 may be configured to communicate with various components of the device 100 and generate a graphical user interface (GUI) to be displayed via a display 128. The controller 120 may include any type of computing device suitable for implementing embodiments of the disclosure. Examples of computing devices include specialized computing devices or general-purpose computing devices such as workstations, servers, laptops, portable devices, desktop, tablet computers, hand-held devices, general-purpose graphics processing units (GPGPUs), and the like, all of which are contemplated within the scope of FIG. 1 with reference to various components of the device 100.

In some embodiments, the controller 120 includes a bus that, directly and/or indirectly, couples the following devices: a processor, a memory, an input/output (I/O) port, an I/O component, and a power supply. Any number of additional components, different components, and/or combinations of components may also be included in the computing device. The bus represents what may be one or more busses (such as, for example, an address bus, data bus, or combination thereof). Similarly, in some embodiments, the computing device may include a number of processors, a number of memory components, a number of I/O ports, a number of I/O components, and/or a number of power supplies. Additionally, any number of these components, or combinations thereof, may be distributed and/or duplicated across a number of computing devices.

In some embodiments, the memory 122 includes computer-readable media in the form of volatile and/or nonvolatile memory, transitory and/or non-transitory storage media and may be removable, nonremovable, or a combination thereof. Media examples include Random Access Memory (RAM); Read Only Memory (ROM); Electronically Erasable Programmable Read Only Memory (EEPROM); flash memory; optical or holographic media; magnetic cassettes, magnetic tape, magnetic disk storage or other magnetic storage devices; data transmissions; and/or any other medium that can be used to store information and can be accessed by a computing device such as, for example, quantum state memory, and/or the like. In some embodiments, the memory 122 stores computer-executable instructions for causing a processor (e.g., the controllers 120) to implement aspects of embodiments of system components discussed herein and/or to perform aspects of embodiments of methods and procedures discussed herein.

The computer-executable instruction 124 may include, for example, computer code, machine-useable instructions, and the like such as, for example, program components capable of being executed by one or more processors associated with a computing device. Program components may be programmed using any number of different programming environments, including various languages, development kits, frameworks, and/or the like. Some or all of the functionality contemplated herein may also, or alternatively, be implemented in hardware and/or firmware.

In some embodiments, the memory 122 may include a data repository implemented using any one of the configurations described below. A data repository may include random access memories, flat files, XML files, and/or one or more database management systems (DBMS) executing on one or more database servers or a data center. A database management system may be a relational (RDBMS), hierarchical (HDBMS), multidimensional (MDBMS), object oriented (ODBMS or OODBMS) or object relational (ORDBMS) database management system, and the like. The data repository may be, for example, a single relational database. In some cases, the data repository may include a plurality of databases that can exchange and aggregate data by data integration process or software application. In an exemplary embodiment, at least part of the data repository may be hosted in a cloud data center. In some cases, a data repository may be hosted on a single computer, a server, a storage device, a cloud server, or the like. In some other cases, a data repository may be hosted on a series of networked computers, servers, or devices. In some cases, a data repository may be hosted on tiers of data storage devices including local, regional, and central.

Various components of the device 100 can communicate via or be coupled to via a communication interface, for example, a wired or wireless interface. The communication interface includes, but not limited to, any wired or wireless short-range and long-range communication interfaces. The wired interface can use cables, umbilicals, and the like. The short-range communication interfaces may be, for example, local area network (LAN), interfaces conforming known communications standard, such as Bluetooth® standard, IEEE 702 standards (e.g., IEEE 702.11), a ZigBee® or similar specification, such as those based on the IEEE 702.15.4 standard, or other public or proprietary wireless protocol. The long-range communication interfaces may be, for example, wide area network (WAN), cellular network interfaces, satellite communication interfaces, etc. The communication interface may be either within a private computer network, such as intranet, or on a public computer network, such as the internet.

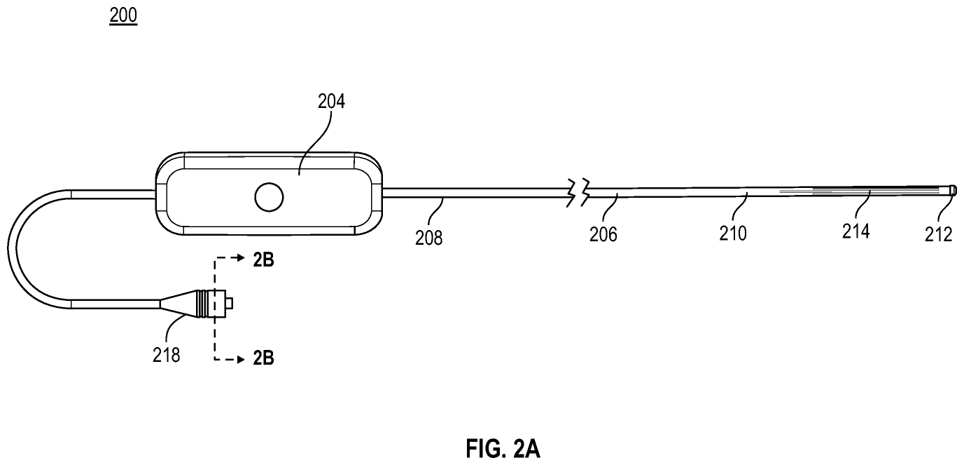

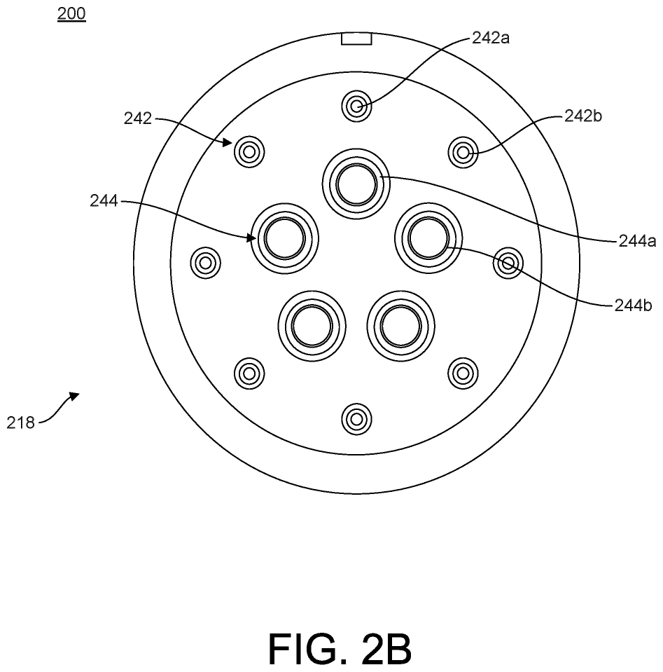

FIG. 2A is a schematic illustration of an exemplary ablation catheter 200 including a connector 218 (similar to the connector 118 as shown in FIG. 1) for treating chronic venous diseases, e.g., varicose veins; FIG. 2B is a cross-sectional view of the connector 218 of the exemplary ablation catheter 200 along the cross-sectional indicator lines 2B-2B of FIG. 2A, according to embodiments of the present disclosure.

As shown, the ablation catheter 200 includes a handle 204, an elongated shaft 206 having a proximal end 208 and a distal end portion 210 terminating at a distal end 212, and an ablation element 214 disposed near the distal end 212 of the elongated shaft 206. The shaft 206 is sized and configured such that the distal end 212 may be inserted into a target blood vessel. The ablation element 214 is configured to deliver ablative energy (e.g., radiofrequency energy, thermal energy) to the wall of a target blood vessel.

In some embodiments, the connector 218 includes pins of different sizes 242 (including e.g., pins 242a, 242b) and 244 (including e.g., pins 244a, 244b). The pins 242 are relatively smaller than pins 244, and are configured to transfer electric signals (e.g., the electric signal generated by the energy generator 116 in FIG. 1). Exemplary electric signals may include thermocouple signals or pressure signals. The pins 244 are relatively larger compared to pins 242, and may be configured to allow current to pass from an energy generator (e.g., the energy generator 116 in FIG. 1) to generate heat on the ablation element 214. One of the pins 244 may be used as a pin connected to ground (i.e., a ground pin). In some embodiments, where the heating elements include multiple heating segments (e.g., individually controllable and/or addressable segments), the ground pin may be used as a common ground pin by the multiple heating segments.

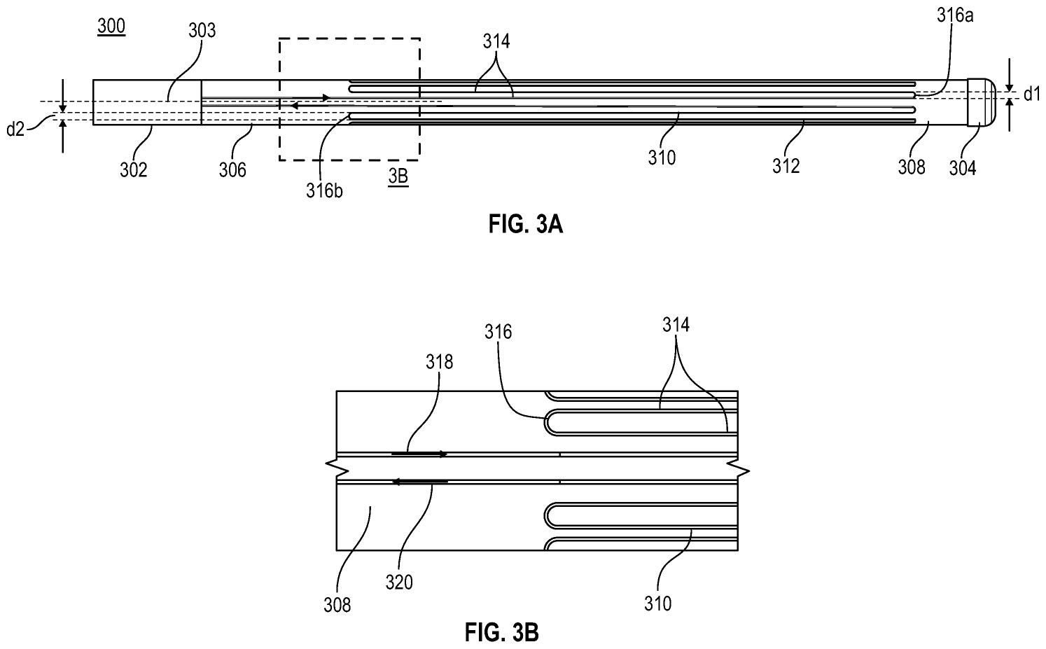

FIG. 3A is a schematic elevation view of the distal end portion 300 of an ablation catheter; FIG. 3B is a partial blown-up view of the distal end portion 300 of an ablation catheter, as indicated by box 3B in FIG. 3A, according to embodiments of the present disclosure. As shown, the distal end portion 300 includes part of an elongated shaft 302 terminating at a distal end 304 and defining a longitudinal axis 303, and an ablation element 306 disposed near the distal end 304 of the elongated shaft 302. The shaft 302 and the ablation element 306 are sized and configured such that the distal end 304 may be inserted into a target blood vessel.

The ablation element 306 includes a tubular polymeric member 308 connected to the elongated shaft 302, and a heating element 310 disposed on the tubular polymeric member 308 and operatively connected to an energy generator (e.g., the energy generator 116 in FIG. 1). In some embodiments, the heating element 310 includes an electrically conductive trace 312 defining a plurality of parallel segments 314 arranged along a length of the tubular polymeric member 308, and an arcuate segment disposed between and connecting the parallel segments 314.

The tubular polymeric member 308 increases the flexibility of the distal portion 300 of an ablation catheter and minimizes potential undesirable harm to vessel walls during treatment. As veins may become tortuous due to chronic venous diseases, it is not easy for operators to insert the distal end portion 300 of an ablation catheter into the target vein. Placement of the ablation element 306 on the distal end portion 300 to a specific treatment site may become increasingly difficult if the catheter is too stiff. Increasing flexibility of the catheter makes it easier for the distal end portion 300 to go through tortuous veins and arrive at target treatment site, and may also reduce the operation time. Diameter, thickness, and material of the tubular polymeric member 308 may be adjusted to further increase the flexibility of the distal portion 300 of an ablation catheter.

A temperature sensor (not shown) may be disposed in the space in between each of the plurality of parallel segments. Based on measured sensor signals indicative temperature from the temperature sensor, a controller (e.g., the energy controller 120 in FIG. 1) or a physician may selectively adjust the power delivery to the heating element 310, thus adjusting heat delivered to the target blood vessel. One or more pressure sensors (not shown) may be disposed proximate to the heating element 310 to measure signals indicative of pressures applied to the heating element 310 via target tissue (e.g., a target vessel wall). In some embodiments, a plurality of pressure sensors (e.g., three sensors, four sensors, six sensors) are disposed circumferentially about the heating element (e.g., two adjacent pressure sensors are offset by certain degrees from one another in a projected view).

In some embodiments, the plurality of pressure sensors include at least one selected from a group consisting of a piezoelectric pressure sensor, a capacitive pressure sensor, an inductive pressure sensor, a strain gauge pressure sensor, and a potentiometric pressure sensor. According to certain embodiments, during treatment, the heating element 310 is controlled to deliver ablative energy when an output signal indicative of pressure generated by at least one pressure sensor of the plurality of pressure sensors is greater than a predetermined threshold. In certain embodiments, the heating element 310 is controlled to deliver ablative energy when output signals indicative of pressure generated by a part of all pressure sensors of the plurality of pressure sensors are greater than a predetermined threshold.

In some embodiments, as shown, the electrically conductive trace 312 includes a plurality of arcuate segments 316, and has a serpentine shape defined by the plurality of parallel segments 314 and the plurality of arcuate segments 316. In embodiments, the parallel segments 314 may be longitudinally or circumferentially spaced from one another on the tubular polymeric member 308, and one of the arcuate segments 316 may be disposed between and connects adjacent parallel segments 314. In some embodiments, the plurality of parallel segments 314 are equally spaced apart.

In an exemplary embodiment, as shown in FIG. 3A-B, the parallel segments 314 extend longitudinally along the longitudinal axis 303 on the tubular polymeric member 308 and are circumferentially spaced from one another. In certain examples, every two adjacent parallel segments connected by an arcuate segment 316a proximate to the distal end 304 have a same spacing d1. In some examples, every two adjacent parallel segments connected by an arcuate segment 316b further away from the distal end 304 have a same spacing d2. In certain examples, the spacing d1 is equal to the spacing d2. In some examples, the spacing d1 is different from the spacing d2. In embodiments, the spacings d1 and d2 between parallel segments may be decreased to increase the density of heating element distribution for higher heat efficiency during treatment. In embodiments, the spacings d1 and d2 between parallel segments may be increased to increase the flexibility of the distal end portion 300 of an ablation catheter depending on specific treatment needs.

In some embodiments, the heating element 310 is disposed on an outer surface of the tubular polymeric member 308. In certain embodiments, the heating element 310 may include segments that are disposed on an inner surface of the tubular polymeric member 308.

During treatment, the electrically conductive trace 312 may receive current delivered by an energy generator (e.g., the energy generator 116 of FIG. 1) traveling in the direction indicated by arrow 318. After the current passes through the plurality of parallel and arcuate segments 314, 316, the current returns in a direction 320 opposite to the initial direction 318. Similarly, due to the serpentine shape, every two adjacent parallel segments have current in opposite directions during treatment. The opposite direction of the current cancels out any magnetic field generated by the current. As inductance may be mostly eliminated by canceling out the magnetic fields, most of the energy generated will be converted into thermal energy rather than electromagnetic energy, thus making treatment more energy efficient.

In an exemplary embodiment, for example as shown in FIG. 3A, one or more pairs of parallel segments 314 are disposed on an outer surface of the tubular polymeric member 308, connected end to end by the arcuate segments 316 with two of the ends connected to a wire that connects the heating element 310 to a generator (e.g., the generator 116 in FIG. 1) through a catheter handle (e.g. the handle 104 in FIG. 1) and a cable (e.g., the cable 105) in FIG. 1. In embodiments, the tubular polymeric member 308 is sized to be inserted into a target vessel while providing ablation efficiency (e.g., sufficiently wide, sufficiently long, etc.). In some embodiments, the length (L) of the tubular polymeric member 308 may be from about three (3) centimeters to about seven (7) centimeters long. In some embodiments, the diameter (d) of the tubular polymeric member 308 may be from about one and a half (1.5) millimeters to about eighteen (18) millimeters. In an exemplary embodiment, the diameter (d) of the tubular polymeric member 308 may be from about one and a half (1.5) millimeters to about one point eight (1.8) millimeters. In certain embodiments, the length of the tubular polymeric member 308 is greater than two (2) centimeters. In some embodiments, the length of the tubular polymeric member 308 is less than ten (10) centimeters. In certain embodiments, the diameter of the tubular polymeric member 308 is greater than one (1) millimeter. In some embodiments, the diameter of the tubular polymeric member 308 is less than twenty (20) millimeters.

In some embodiments, the heating element 310 includes a Ni—Cr alloy or a carbon film. The Ni—Cr alloy or the carbon film may be electroplated or sprayed onto the tubular polymeric member 308. In some embodiments, the electroplated or sprayed film may be from about 0.05 μm to about 0.3 μm. In an exemplary embodiment, the electroplated or sprayed film may be from about 0.1 μm to about 0.2 μm.

Compared to a heating element that uses coils (e.g., resistance wires), one of the benefits of electroplating or metal spraying the film is the increase of consistency in manufacturing method to achieve a more even and/or smooth heating surface. The film may be electroplated or metal sprayed, or produced using any method commonly used for producing flexible circuits as understood by a skilled artisan. Although the film may be produced using a method similar to flexible circuits, in some embodiments, the film includes material that has electrical resistance larger than typical materials used for flexible circuits. In embodiments, the heating element 310 including the sprayed or electroplated film is operatively connected to an energy generator (e.g., the generator 116 in FIG. 1) and configured to generate thermal energy in response to receiving an electric signal from the energy generator.

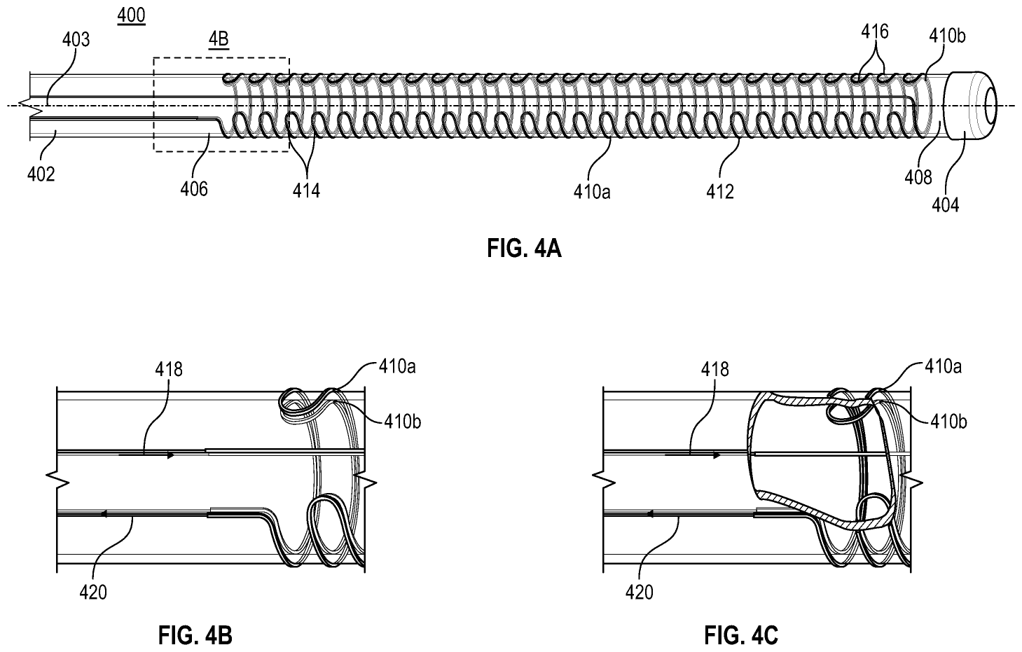

FIG. 4A is a phantom schematic elevation view of a distal end portion 400 of an ablation catheter; FIG. 4B is a partial blown up view of a distal end portion 400 of an ablation catheter, as indicated by box 4B in FIG. 4A; FIG. 4C is a cutaway view of the partial blown-up view shown in FIG. 4B of a distal end portion 400 of an ablation catheter, according to embodiments of the present disclosure. As shown, the distal end portion 400 includes part of an elongated shaft 402 terminating at a distal end 404 and defining a longitudinal axis 403, and an ablation element 406 disposed near the distal end 404 of the elongated shaft 402. The shaft 402 and the ablation element 406 are sized and configured such that the distal end 404 may be inserted into a target blood vessel.

The ablation element 406 includes a tubular polymeric member 408 connected to the elongated shaft 402, and a heating element 410 disposed on the tubular polymeric member 408 and operatively connected to an energy generator (e.g., the energy generator 116 in FIG. 1). In some embodiments, the heating element 410 includes an electrically conductive trace 412 defining a plurality of parallel segments 414 arranged along a length of the tubular polymeric member 408, and one or more arcuate segments disposed between and connecting the parallel segments 414.

The tubular polymeric member 408 increases the flexibility of the distal portion 400 of an ablation catheter and minimizes potential undesirable harm to vessel walls during treatment. A temperature sensor (not shown) may be disposed in the space in between each of the plurality of parallel segments. Based on measured signals indicative of temperature by the temperature sensor, a controller (e.g., the energy controller 120 in FIG. 1) or a physician may adjust the power delivery to the heating element 410, thus adjusting thermal ablative energy delivered to the target blood vessel.

In some embodiments, as shown, the electrically conductive trace 412 includes a plurality of arcuate segments 416, and has a serpentine shape defined by the plurality of parallel segments 414 and the plurality of arcuate segments 416.

In embodiments, the parallel segments 414 may be longitudinally or circumferentially spaced from one another on the tubular polymeric member 408, and one of the arcuate segments 416 may be disposed between and connects adjacent parallel segments 414. In some embodiments, each of the plurality of parallel segments 414 are equally spaced apart. In an exemplary embodiment, for example as shown in FIGS. 4A-4C, the parallel segments 414 extend circumferentially about the tubular polymeric member 408 and are longitudinally spaced from one another along the longitudinal axis 403.

In some embodiments, the heating element 410a is disposed on an outer surface of the tubular polymeric member 408. In certain embodiments, for example as shown in FIGS. 4A-4C, the heating element 410b may include segments that are disposed on an inner surface of the tubular polymeric member 408. In an exemplary embodiment, the heating element 410 includes both 410a and 410b disposed on the outer surface and inner surface, respectively, of the tubular polymeric member 408.

During treatment, the electrically conductive trace 412 may receive current delivered by an energy generator (e.g., the energy generator 116 of FIG. 1) traveling in the direction indicated by arrow 418. After the current passes through the plurality of parallel and arcuate segments 414, 416, the current returns in a direction 420 opposite to the initial direction 418. Similarly, due to the serpentine shape, each adjacent parallel segment 414 has current going through the segment during treatment that are in opposite directions. The opposite direction of the current cancels out any magnetic field generated by the current, thus making treatment more energy efficient. In some embodiments, the heating element 410b disposed on the inner surface of the tubular polymeric member 408 has current going through it in the opposite direction of current going through the heating element 410a disposed on the outer surface of the tubular polymeric member 408, further canceling out the magnetic field generated by the current during treatment. In embodiments where the heating element 410b has current in opposite direction to current going through heating element 410a, heating element 410a may act as a therapeutically active heating element, and heating element 410b may act as a therapeutically passive heating element for countering the electric field generated by current running through the therapeutically active heating element 410a. As inductance may be mostly eliminated by canceling out the magnetic fields, most of the energy generated will be converted into thermal energy rather than electromagnetic energy, thus making treatment more energy efficient.

In some embodiments, the heating element 410 includes a Ni—Cr alloy or a carbon film. The Ni—Cr alloy or the carbon film may be electroplated or sprayed onto the tubular polymeric member 408.

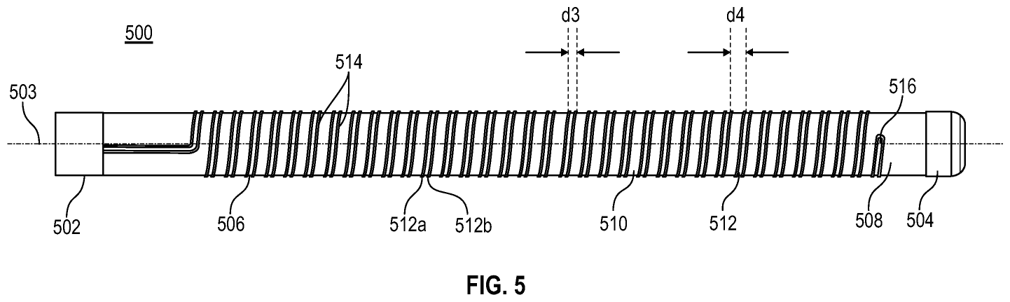

FIG. 5 is a schematic illustration of the distal end portion of an ablation catheter, according to embodiments of the present disclosure.

As shown, the distal end portion 500 includes part of an elongated shaft 502 terminating at a distal end 504 and defining a longitudinal axis 503, and an ablation element 506 disposed near the distal end 504 of the elongated shaft 502. The shaft 502 and the ablation element 506 are sized and configured such that the distal end 504 may be inserted into a target blood vessel.

The ablation element 506 includes a tubular polymeric member 508 connected to the elongated shaft 502, and a heating element 510 disposed on the tubular polymeric member 508 and operatively connected to an energy generator (e.g., the energy generator 116 in FIG. 1). In some embodiments, the heating element 510 includes an electrically conductive trace 512 defining a plurality of parallel segments 514 arranged along a length of the tubular polymeric member 508 and an arcuate segment 516 disposed between and connecting two adjacent parallel segments of the plurality of parallel segments 514. The tubular polymeric member 508 increases the flexibility of the distal portion 500 of an ablation catheter and minimizes potential undesirable harm to vessel walls during treatment. A temperature sensor (not shown) may be disposed in the space in between each of the plurality of parallel segments 514. Based on measured signals indicative of a temperature from the temperature sensor, a controller (e.g., the energy controller 120 in FIG. 1) or a physician may selectively adjust the power delivery to the heating element 510, thus adjusting heat delivered to the target blood vessel. In some embodiments, the power delivery to the heating element 510 may heat the heating element 510 to about 80° C. to about 140° C. for treating varicose veins. In some embodiments, the power delivery to the heating element 510 may heat the heating element 510 to about 100° C. to about 130° C. for treating varicose veins. In some embodiments, the power delivery to the heating element 510 may heat the heating element 510 to about 120° C. for treating varicose veins.

In an exemplary embodiment, as shown in FIG. 5, the parallel segments 514 in the conductive trace 512 are helically wound about the tubular polymeric member 508. In some embodiments, the conductive trace 512 includes two sections 512a and 512b, where the two sections 512a and 512b are parallel and form a pair pattern. In certain examples, the section pair has a spacing d3. In some examples, the spacing between two adjacent section pairs is d4. In certain examples, the spacing d3 is equal to the spacing d4. In some examples, the spacing d3 is different from the spacing d4.

In some embodiments, the heating element 510 is disposed on an outer surface of the tubular polymeric member 508. In certain embodiments, the heating element 510 may include segments that are disposed on an inner surface of the tubular polymeric member 508. In some embodiments, the heating element disposed on the inner surface (not shown) of the tubular polymeric member 508 has current going through it in the opposite direction of current going through the heating element 510 disposed on the outer surface of the tubular polymeric member 508, canceling out the magnetic field generated by the current going through each of the heating element 510 during treatment. In some embodiments, the heating element 510 includes a Ni—Cr alloy or a carbon film. The Ni—Cr alloy or the carbon film may be electroplated or sprayed onto the tubular polymeric member 508.

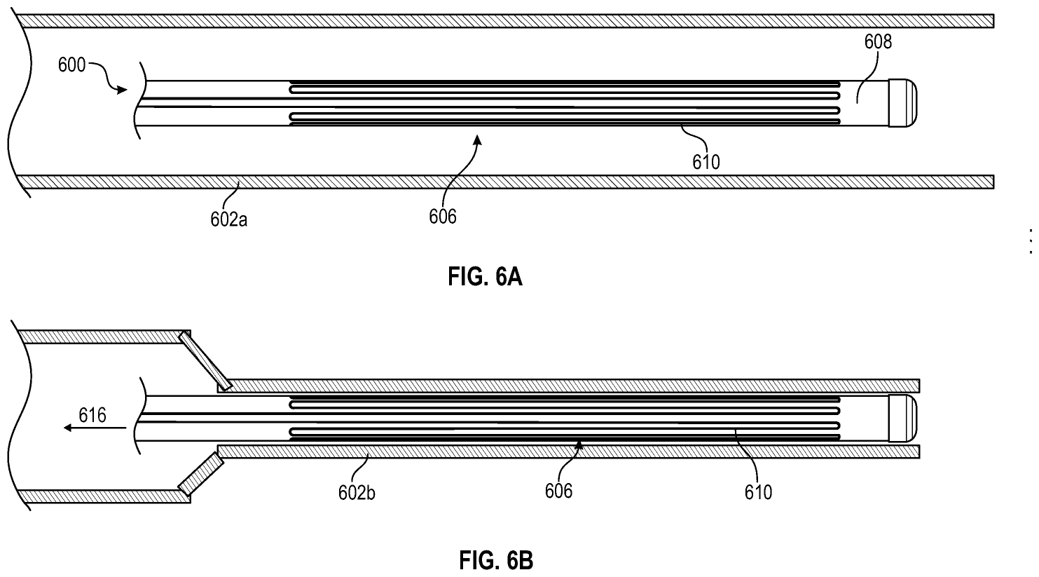

FIGS. 6A-6B are schematic illustrations of a portion of an ablation catheter for use in a target blood vessel in a patient for treatment of varicose veins, according to embodiments of the present disclosure.

In some embodiments, during endovenous thermal ablation procedure, an introducer sheath may be positioned inside a patient's target vein using ultrasonic guidance and standard vascular technique. An ablation catheter (e.g., the ablation catheter 102 in FIG. 1) may then be inserted into the target vein through the introducer sheath. In some circumstances, under ultrasonic guidance, tumescent anesthetic solution or saline may be injected into target vein segment to act as a heat sink that protects tissue from thermal injury, and improve thermal conductivity between the wall of target vein and the ablation catheter.

As shown in FIG. 6A, the distal end portion 600 of an ablation catheter (e.g., the ablation catheter 102 in FIG. 1) is positioned in a target blood vessel 602a. The ablation catheter may be introduced and positioned with an introducer sheath using ultrasonic guidance. As will be appreciated by a skilled artisan, any standard vascular technique may be used here to introduce and position the distal end portion 600 of the ablation catheter into the target vein segment. The distal end portion 600 may include an ablation element 606 having a tubular polymeric member 608 and a heating element 610 disposed on the tubular polymeric member 608 and operatively connected to an energy generator (e.g., the energy generator 116 in FIG. 1).

In some embodiments, during treatment, current may be applied to the heating element 610 by a generator (e.g., the energy generator 116 in FIG. 1), and a segment of the target blood vessel 602a adjacent the heating element 610 being treated will close as the heating element 610 is heated up, shown as 602b in FIG. 6B. The generator may include a radiofrequency generator that generates radiofrequency current to heat the heating element 610. In some implementations, the ablation catheter may include a temperature sensor disposed along the length of a shaft of the catheter, and power delivery to the heating element 610 may be adjusted automatically by a controller (e.g., the controller 120 in FIG. 1) based on temperature or signals indicative of temperature measured by the temperature sensor.

A segment of the target blood vessel 602a adjacent the heating element 610 being treated will close (e.g., shrink, reduced in diameter) as the heating element 610 is heated up, shown as 602b in FIG. 6B. External pressure may be applied as needed during treatment. After a certain section is treated (i.e. the section of the vein is closed), the catheter may be moved towards the venous access, as indicated by arrow 616, and the process repeated until the entire vein is closed. The catheter and introducer sheath may then be removed after treatment is done. In some use cases, a diameter of the ablation element 606 is smaller than a diameter of blood vessel 602a and the ablation element 606 can be moved close to the vessel wall during the treatment.

In Example 16, a device for treating varicose veins includes an energy generator configured to generate an electric signal; a controller operatively connected to the energy generator to control the generation of the electric signal; and a catheter connected to the energy generator. The catheter includes an elongated shaft having a proximal end and a distal end, the shaft being sized and configured such that the distal end can be inserted into a target blood vessel; and an ablation element disposed near the distal end of the elongated shaft. In some embodiments, the ablation element includes a tubular polymeric member connected to the elongated shaft; and a heating element disposed on the tubular polymeric member, the heating element comprising an electrically conductive trace defining a plurality of parallel segments arranged along a length of the tubular polymeric member and one or more arcuate segments, at least one of the one or more arcuate segments disposed between and connecting two adjacent parallel segments of the plurality of parallel segments, wherein the electrically conductive trace is operatively connected to the energy generator and configured to generate ablative energy in response to receiving the electric signal from the energy generator.

In Example 17, the device of Example 13, wherein the electrically conductive trace has a serpentine shape defined by the plurality of parallel segments and the one or more arcuate segments, wherein the parallel segments are longitudinally or circumferentially spaced from one another on the tubular polymeric member.

In Example 18, the device of Example 14, wherein the plurality of parallel segments extend longitudinally along the tubular polymeric member and are circumferentially spaced from one another.

In Example 19, the device of Example 14, wherein the plurality of parallel segments extend circumferentially about the tubular polymeric member and are longitudinally spaced from one another.

In Example 20, the device of Example 13, wherein the plurality of parallel segments are helically wound about the tubular polymeric member.

In Example 21, the device of any of Examples 13-17, wherein the heating element is disposed on an outer surface of the tubular polymeric member.

In Example 22, the device of any of Examples 13-17, wherein at least a part of the heating element is disposed on an inner surface of the tubular polymeric member.

In Example 23, the device of any of Examples 13-19, wherein the heating element comprises a Ni—Cr alloy.

In Example 24, the device of any of Examples 13-19, wherein the heating element comprises a carbon film.

In Example 25, the device of any of Examples 13-21, wherein the plurality of parallel segments are equally spaced apart.

In Example 26, the device of Example 20, wherein the Ni—Cr alloy is electroplated or spayed onto the tubular polymeric member.

In Example 27, the device of Example 21, wherein the carbon film is electroplated or sprayed onto the tubular polymeric member.

As the terms are used herein with respect to measurements (e.g., dimensions, characteristics, attributes, components, etc.), and ranges thereof, of tangible things (e.g., products, inventory, etc.) and/or intangible things (e.g., data, electronic representations of currency, accounts, information, portions of things (e.g., percentages, fractions), calculations, data models, dynamic system models, algorithms, parameters, etc.), “about” and “approximately” may be used, interchangeably, to refer to a measurement that includes the stated measurement and that also includes any measurements that are reasonably close to the stated measurement, but that may differ by a reasonably small amount such as will be understood, and readily ascertained, by individuals having ordinary skill in the relevant arts to be attributable to measurement error; differences in measurement and/or manufacturing equipment calibration; human error in reading and/or setting measurements; adjustments made to optimize performance and/or structural parameters in view of other measurements (e.g., measurements associated with other things); particular implementation scenarios; imprecise adjustment and/or manipulation of things, settings, and/or measurements by a person, a computing device, and/or a machine; system tolerances; control loops; machine-learning; foreseeable variations (e.g., statistically insignificant variations, chaotic variations, system and/or model instabilities, etc.); preferences; and/or the like.

Although illustrative methods may be represented by one or more drawings (e.g., flow diagrams, communication flows, etc.), the drawings should not be interpreted as implying any requirement of, or particular order among or between, various steps disclosed herein. However, certain some embodiments may require certain steps and/or certain orders between certain steps, as may be explicitly described herein and/or as may be understood from the nature of the steps themselves (e.g., the performance of some steps may depend on the outcome of a previous step). Additionally, a “set,” “subset,” or “group” of items (e.g., inputs, algorithms, data values, etc.) may include one or more items, and, similarly, a subset or subgroup of items may include one or more items. A “plurality” means more than one.

Various modifications and additions can be made to the exemplary embodiments discussed without departing from the scope of the present invention. For example, while the embodiments described above refer to particular features, the scope of this invention also includes embodiments having different combinations of features and embodiments that do not include all of the described features. Accordingly, the scope of the present invention is intended to embrace all such alternatives, modifications, and variations as fall within the scope of the claims, together with all equivalents thereof.

1. A device for treating varicose veins, comprising:
a catheter comprising:
an elongated shaft having a proximal end and a distal end, the shaft being sized and configured such that the distal end can be inserted into a target blood vessel; and
an ablation element disposed near the distal end of the elongated shaft, the ablation element comprising:
a tubular polymeric member connected to the elongated shaft; and
a heating element disposed on the tubular polymeric member, the heating element comprising an electrically conductive trace defining a plurality of parallel segments arranged along a length of the tubular polymeric member and one or more arcuate segments, at least one of the one or more arcuate segments disposed between and connecting two adjacent parallel segments of the plurality of parallel segments.

2. The device of claim 1, wherein the electrically conductive trace has a serpentine shape defined by the plurality of parallel segments and the one or more arcuate segments, wherein the plurality of parallel segments are longitudinally or circumferentially spaced from one another on the tubular polymeric member.

3. The device of claim 2, wherein the plurality of parallel segments extend longitudinally along the tubular polymeric member and are circumferentially spaced from one another.

4. The device of claim 2, wherein the plurality of parallel segments extend circumferentially about the tubular polymeric member and are longitudinally spaced from one another.

5. The device of claim 1, wherein the plurality of parallel segments are helically wound about the tubular polymeric member.

6. The device of claim 1, wherein the heating element is disposed on an outer surface of the tubular polymeric member.

7. The device of claim 1, wherein at least a part of the heating element is disposed on an inner surface of the tubular polymeric member.

8. The device of claim 1, wherein the heating element comprises a Ni—Cr alloy.

9. The device of claim 1, wherein the heating element comprises a carbon film.

10. The device of claim 1, wherein the plurality of parallel segments are equally spaced apart.

11. The device of claim 8, wherein the Ni—Cr alloy is electroplated or spayed onto the tubular polymeric member.

12. The device of claim 9, wherein the carbon film is electroplated or sprayed onto the tubular polymeric member.

13. A system for treating varicose veins, comprising:
a catheter comprising:
an elongated shaft having a proximal end and a distal end, the shaft being sized and configured such that the distal end can be inserted into a target blood vessel; and
an ablation element disposed near the distal end of the elongated shaft, the ablation element comprising:
a tubular polymeric member connected to the elongated shaft; and
a heating element disposed on the tubular polymeric member, the heating element comprising an electrically conductive trace defining a plurality of parallel segments arranged along a length of the tubular polymeric member and one or more arcuate segments, at least one of the one or more arcuate segments disposed between and connecting two adjacent parallel segments of the plurality of parallel segments;
an energy generator connected to the elongated catheter and configured to generate an electric signal; and
a controller operatively connected to the energy generator to control the generation of the electric signal.

14. The system of claim 13, wherein the heating element is electrically coupled to the energy generator.

15. The system of claim 13, wherein the device includes one or more pressure sensors configured to generate an output signal indicative of a pressure applied thereto, and wherein the controller is configured to adjust the electric signal to be delivered to the heating element based upon the output signal.

16. A catheter for treating varicose veins, comprising:
an elongated shaft having a proximal end and a distal end; and
an ablation element disposed near the distal end of the elongated shaft, the ablation element comprising:
a tubular polymeric member connected to the elongated shaft; and
a heating element disposed on the tubular polymeric member, the heating element comprising an electrically conductive trace defining a plurality of parallel segments arranged along a length of the tubular polymeric member, and a plurality of arcuate segments, wherein a respective arcuate segment of the plurality of arcuate segments is disposed between and connects two adjacent parallel segments of the plurality of parallel segments.

17. The catheter of claim 16, wherein the electrically conductive trace has a serpentine shape defined by the plurality of parallel segments and the one or more arcuate segments, wherein the plurality of parallel segments are longitudinally or circumferentially spaced from one another on the tubular polymeric member.

18. The catheter of claim 17, wherein the plurality of parallel segments extend longitudinally along the tubular polymeric member and are circumferentially spaced from one another.

19. The catheter of claim 17, wherein the plurality of parallel segments extend circumferentially about the tubular polymeric member and are longitudinally spaced from one another.

20. The catheter of claim 16, wherein the plurality of parallel segments are helically wound about the tubular polymeric member.
